(19) United States
(12) Patent Application Publication
(10) Pub. No.: US 2023/0330169 A1
(43) Pub. Date: Oct. 19, 2023

(54) PHARMACEUTICAL COMPOSITIONS

(71) Applicant: PROTA THERAPEUTICS PTY LTD, Melbourne (AU)

(72) Inventors: Bronwyn POLLOCK, Blackburn South (AU); Sharon HANEGRAAF, Wantirna South (AU); Digant BODAWALA, Whippany, NJ (US); Satish SHETTY, Morris Plains, NJ (US)

(21) Appl. No.: 17/788,198

(22) PCT Filed: Apr. 24, 2020
(86) PCT No.: PCT/AU2020/050400

§ 371 Date: Jun. 22, 2022

(30) Foreign Application Priority Data
Dec. 23, 2019 (EP) 19219500.6

(51) Int. Cl. A61K 36/48; A61K 9/20; A61K 45/06

(52) U.S. Cl. CPC A61K 36/48; A61K 9/2018; A61K 9/2009; A61K 9/2059; A61K 9/2013; A61K 9/2054; A61K 45/06

(57) ABSTRACT

The present invention relates to pharmaceutical compositions comprising peanut protein, methods of manufacturing such pharmaceutical compositions and uses for such pharmaceutical compositions. Embodiments of the invention relate to pharmaceutical peanut protein compositions for oral administration in immunotherapy for the treatment of peanut allergy.

### FIELD

The present invention relates to pharmaceutical compositions comprising peanut protein, methods of manufacturing such pharmaceutical compositions and uses for such pharmaceutical compositions. Embodiments of the invention relate to pharmaceutical peanut protein compositions for oral administration in immunotherapy for the treatment of peanut allergy.

### BACKGROUND

Food allergies, or the body's immunological reaction to allergens from proteins in food, can severely impact the quality of life for sufferers. It is estimated that 5%-6% of children in Western societies have food allergy, and 1.5% of children have peanut allergy.

Reactions to peanut may be severe and peanut allergy is responsible for between 19% and 63% of deaths from food induced anaphylaxis. Unlike other food allergies, peanut allergy is usually lifelong. Current management of food allergy requires strict avoidance of the food; however, 50% of peanut allergic patients are accidentally exposed to peanut within 2 years. Therefore, children with peanut allergy are at continued risk of severe life-threatening allergic reactions throughout their lifetime.

The current standard of care for food allergy is a strict elimination diet to avoid exposure to an allergen, combined with the early treatment of allergic reactions, particularly anaphylaxis, in the event of accidental exposure to an allergen. Adrenaline is the first line therapy for anaphylaxis and is available as a self-injectable device (the EpiPen®), which is generally carried by persons with severe food allergy at all times. However, this requires specialist training in its use, devices must be replaced regularly (12-18 months), and the majority of patients who have been prescribed an EpiPen® fail to use it at a time of severe allergic reaction: of patients prescribed an EpiPen®, only 71% had it with them at the time, and of those, 10% had expired devices and only 32% were able to demonstrate its correct use.

A number of immunotherapy approaches have been explored for the treatment of food allergy. These typically involve the regular and gradual exposure of a subject to increasing doses of an allergen to which they are allergic. There are a number of treatment outcomes that can be achieved with these various treatments. For example, desensitization is defined as a transient increase in the threshold dose required to trigger an allergic reaction. This protection is only able to be maintained if there is continuing regular exposure to the food allergen, such as with continued treatment. Sustained unresponsiveness is defined as the sustained ability to ingest a food allergen without reaction that is maintained without the need for continuing regular allergen exposure. This may reflect a state of tolerance, defined as the ability to eat any amount of allergen ad libitum without reaction and without the need for continuing regular allergen exposure. In terms of clinical benefit to the patient, desensitization offers protection against accidental exposure to small amounts of allergen provided the treatment is continued indefinitely, whereas sustained unresponsiveness or tolerance offer long-lasting protection without the need for continuing treatment thus enabling the patient to incorporate the allergen into their normal diet if they so wish.

Immunotherapy with the allergen to which the subject is allergic may be administered subcutaneously (SCIT), sublingually (SLIT), epicutaneously (EIT) or orally (OIT).

Typically, OIT with food allergens (i.e. for the treatment of food allergy) involves orally administering regular doses of the food allergen in increasing amounts to food allergic subjects, starting with an initial very small dose, extremely unlikely to cause an adverse reaction, escalating over time to a maintenance dose which is administered regularly over several months or years or indefinitely.

Studies have shown that OIT with egg, milk, peanut or other food allergens can induce desensitization with accompanying reduced skin prick test (SPT), and increased serum levels of allergen specific IgG4.

Combined administration of a probiotic and food allergen oral immunotherapy has been shown to result in high rates of sustained unresponsiveness (see WO2009/094717).

OIT protocols typically include three phases of treatment—rush (also referred to as initiation), build-up and maintenance. Together the rush phase and build-up phase form the dose escalation phase. The rush phase generally involves administering a series of escalating allergen doses (e.g. 6-8 doses) over one day, starting at a very low dose that the subject can tolerate, often as low as 0.1 mg. The build-up phase generally involves administering a daily dose of the food allergen, typically commencing at the highest tolerated dose of the rush phase (or the cumulative tolerated dose in the rush phase if all rush phase doses are tolerated), with increases in the dose every 1-2 weeks. The maintenance phase continues the treatment at the highest dose reached during build-up phase and provides the same dose of the food allergen daily, often for months to years or indefinitely. The highest dose may be 1000 mg or more.

The first regulatory approval for a food immunotherapy treatment was given by the FDA in early 2020 to AR101 (PALFORZIA™), a peanut oral immunotherapy developed by Aimmune Therapeutics, Inc. AR101 (Palforzia) is a characterized peanut flour preparation formulated to deliver standardized allergen doses within pull apart capsules. Its use in a phase 2 peanut OIT clinical trial (ARC001) is described in Bird, J. A, et al., (2018) JACI Pract. 6:476-85. ARC001 involves daily dosing of AR101 or placebo, gradually updosed from 0.5 to 300 mg/day. Other OIT clinical trials have administered peanut allergens as uncharacterized food grade peanut flour, crushed or ground peanuts, as peanut butter or in a chocolate bar.

While the levels of allergens in peanut has been shown to be stable over time (to at least 18 months), the use of food products in immunotherapy is not ideal as it is difficult to measure small quantities of these products, they may soil or go rancid over time, depending on the fat component, and in the case of peanut flour, may clump or adhere to packaging, preventing accurate administration of low allergen doses which are required for initial phases of OIT, particularly in the rush phase. Additionally, to obtain regulatory approval in the USA with the Food & Drug Administration (FDA), the submission of a Biologics License Application is required, comprising detailed documentation of the characteristics of the peanut formulation, which must also be manufactured in a Good Manufacturing Practices (GMP)-compliant environment under Standard Operating Procedures (SOPs) detailing the process.

AR101 (PALFORZIA™) is a defatted peanut flour formulation presented in pull-apart capsules. Administration involves pulling apart the capsule (which may be difficult to manage) and then mixing the contents with a vehicle food for ingestion. The act of pulling apart the capsule may result in the partial loss of the dose to be administered resulting in inaccurate administration in small quantities. Further, when pulling apart the capsule, the peanut flour may be dispersed into the air, carrying a risk of inhalation of the powder and therefore a risk of inducing extreme reactions. Further, encapsulation introduces limitations on the quantity of peanut flour and therefore peanut protein which can be included in any single dose, posing challenges for delivery of higher doses of peanut allergen. In addition, peanut flour is hygroscopic which creates additional challenges in formulating a long shelf life product.

There exists a need to provide a pharmaceutical peanut flour formulation for use in OIT to treat peanut allergy, in which the pharmaceutical formulation can be easily and safely administered in small or large doses with accuracy. To avoid airborne peanut flour and the associated risk for peanut allergens to be inhaled, it is preferred that the peanut flour formulations are in the form of granules, tablets or mini-tablets. However, making such formulations requires the pharmaceutical peanut flour formulation to have acceptable flow and binding properties such that it may be compressed into a granule, tablet or mini-tablet with appropriate hardness and acceptable handling properties during manufacture and packaging. At the same time, to avoid potential choking hazard for infants and young children, the formulation should readily disintegrate when mixed with age appropriate food vehicles (e.g. custard, apple puree, puddings), allowing safe ingestion in infants and young children.

It is an aim of a preferred embodiment of the present invention to provide a pharmaceutical peanut flour formulation that satisfies some or all these needs.

### SUMMARY

The present inventors have developed new peanut flour formulations with acceptable flow and binding properties to allow them to be formulated into pharmaceutical compositions that can be used in oral immunotherapy (OIT) for subjects that are allergic to peanuts. The formulations have acceptable handling properties during manufacture and packaging and may be compressed into a granule, tablet or mini-tablet with accurate dosing and appropriate hardness. Further, the formulations have acceptable disintegration times when mixed in water or soft foods or may be crushed to assist in disintegration to avoid a choking hazard.

In one aspect the present invention provides a pharmaceutical composition comprising peanut flour, at least one disintegrant, at least one adsorbent, at least one diluent and at least one lubricant, wherein the peanut flour comprises about 40% to about 60% peanut protein.

In one embodiment the pharmaceutical composition further comprises a probiotic.

In a preferred embodiment the pharmaceutical composition is a granule, tablet or mini-tablet formulation.

Another aspect of the invention provides a method of treating peanut allergy comprising administering the pharmaceutical composition of the invention, optionally by oral immunotherapy.

A further aspect of the invention provides the composition of the invention for use in treating peanut allergy, optionally by oral immunotherapy.

A yet further aspect of the invention provides the use of peanut flour, at least one disintegrant, at least one adsorbent, at least one diluent and at least one lubricant, wherein the peanut flour comprises 40% to 60% peanut protein, in the manufacture of a medicament for treating peanut allergy, optionally by oral immunotherapy.

In one embodiment the method, composition for use or use further comprises administering either simultaneously or sequentially, a probiotic.

In yet another aspect of the invention there is provided a kit comprising at least two doses of a composition of the invention, each dose package separately.

In one embodiment the kit further comprises a probiotic, optionally packaged separately to the pharmaceutical composition.

In yet another aspect of the invention there is provided a method of making the composition of the invention comprising the steps of:
a. blending peanut flour with at least one adsorbent; wherein the peanut flour comprises 40% to 60% peanut protein;
b. mixing at least one diluent and at least one disintegrant with the blend from step a;
c. adding at least one lubricant to the mixture of step b. and mixing.

Optionally any other excipients are added at step b.

The method may further comprise formulating the mixture from step c. into granules, tablets or mini-tablets

### DETAILED DESCRIPTION OF THE INVENTION

The present invention relates to peanut flour compositions and their use in treating peanut allergy. In one aspect of the present invention there is provided a pharmaceutical composition comprising peanut flour, at least one disintegrant, at least one adsorbent, at least one diluent and at least one lubricant, wherein the peanut flour comprises about 40% to about 60% peanut protein.

Peanuts are widely known to comprise approximately 25% w/w peanut protein, whereas peanut flour is known to comprise approximately 50% w/w peanut protein (although the precise values may vary depending on the source of the peanut and factors such as whether the peanut has been processed in any way). Thus, by way of representative example, a dose of 2000 mg of peanut protein may be the equivalent of approximately 8000 mg peanuts (approximately eight to ten peanuts). The peanut flour used in the composition of the present invention has about 50% w/w peanut protein, for example in the range of about 40% to about 60% w/w of the peanut flour, for example, 45% to 55% w/w of the peanut flour or 48% to 52% w/w of the peanut flour. The protein content of the peanut flour is determined by known methods such as acid digestion followed by titrimetric measurement of nitrogen content (nitrogen determination by the Kjeldahl Method described in United States Pharmacopeia General Chapter 461 Nitrogen Determination). In some embodiments, the peanut flour is defatted to provide flour with about 10 to 15% fat w/w, especially about 12% fat w/w. A suitable peanut flour product is supplied by Byrd Mill (Ashland, VA, USA), however other food grade peanut flours which are defatted and contain approximately 50% w/w peanut protein could also be used.

Peanut flour generally comprises peanut proteins Ara h1, Ara h2, Ara h3, Ara h4, Ara h5, Ara h6, Ara h7, Ara h8 and Ara h9 to various degrees. To obtain regulatory approval in the USA with the Food & Drug Administration (FDA), the submission of a Biologics License Application is required, comprising detailed documentation of the characteristics of the peanut formulation, which must also be manufactured in a Good Manufacturing Practices (GMP)-compliant environment under Standard Operating Procedures (SOPs) detailing the process. It may be important to quantify one or more of Ara h1, Ara h2, Ara h3 and Ara h6 in the peanut flour used in the composition and this may be achieved using methods well known in the art, such as reverse phase HPLC.

In the manufacturing process, the composition is prepared using a procedure called potency adjustment. This involves calculating the quantity of peanut flour required based on the protein content of the batch of peanut flour. The quantity of diluent (for example, mannitol) required is adjusted based on the peanut flour quantity required to provide the correct protein content.

In some embodiments, the peanut protein is present in the composition in an amount of about 1% to about 60% w/w or about 5% to about 60% w/w of the composition, especially about 20% to about 60% w/w of the composition, more especially about 20% to about 50% w/w of the composition, even more especially 20% to about 40% or 20% to about 30% w/w of the composition. In other embodiments, the composition may comprise peanut flour in an amount from about 1% to about 30% w/w of the composition, especially about 1% to about 40% w/w of the composition, for example, 1% to 10% w/w or 20% to 40% w/w of the composition or 5-10% or 20-30% of the composition.

In some embodiments, the peanut protein is present in the composition in an amount of 0.1 to 4000 mg, especially about 0.1 to 2000 mg, more especially about 0.1 to 1000 mg, even more especially 0.1 to 100 mg, even more especially 0.1 to 10 mg. Particular compositions comprise 0.1 mg peanut protein, 0.4 mg peanut protein and 2.0 mg peanut protein.

Pharmaceutical compositions generally comprise one or more excipients. Pharmaceutical excipients are substances other than the pharmacologically active drug or prodrug which are included in the manufacturing process or are contained in a finished pharmaceutical product dosage form and include subgroups such as diluents or fillers, binders or adhesives, adsorbents, disintegrants, lubricants, glidants, flavors, colors and sweeteners. The selection of excipients for a particular formulation involves consideration of a number of complex factors including the nature of the active pharmaceutical ingredient (API), to avoid any excipients which may react, and the required properties of the final dosage form including adhesion, friability, disintegration, location and timing of delivery (e.g. through the gut or extended release formulations). Excipients also commonly have more than one function, for example, talc is commonly characterised as one or more of an anti-caking agent, glidant, tablet and capsule diluent or tablet and capsule lubricant.

Adsorbents are sometimes used in tableting processes to absorb water and/or fats from the active pharmaceutical ingredient. Due to its moisture-scavenging properties, the quantity of adsorbent used can critically affect the hardness and friability of the resulting tablet or granule.

The composition may comprise one or more adsorbents, such as silicon dioxide e.g. Syloid® XDP 3050 (Grace), synthetic amorphous silica or colloidal silicon dioxide (fumed silica) such as Aerosil® 200 pharma (Evonik), phyllosilicates, kaolinite, magnesium carbonate, cellulose, microcrystalline cellulose and sodium carboxy methyl cellulose.

In some embodiments, the adsorbent is present in an amount of about 1% to about 45% w/w of the composition, especially about 5% to about 40% w/w of the composition or about 11% to about 40% w/w of the composition, more especially about 5% to about 9% of the composition, 15% to about 35% w/w of the composition, or about 20% to about 35% w/w of the composition. In some embodiments, the amount of adsorbent is between about 14% and 40% w/w of the composition.

The pharmaceutical composition may also comprise one or more disintegrants that allows the composition to dissolve into water or disperse into food. There are also a sub-class of disintegrants known as superdisintegrants. These substances are more effective disintegrants at lower concentrations with greater disintegrating efficiency and mechanical strength. On contact with water the superdisintegrants swell, hydrate, change volume or form and produce a disruptive change in the tablet. Effective superdisintegrants provide improved compressibility, compatibility and have no negative impact on the mechanical strength of formulations containing high-dose drugs. Suitable disintegrants include low-substituted hydroxy propyl cellulose such as L-HPC (Shin-Estu), sorbitol such as Neosorb® P60W (Roquette), citric acid, carboxymethylcellulose calcium, microcrystalline cellulose or a mixture thereof. Suitable superdisintegrants include sodium starch glycolate, for example Primojel® (JRS Pharma), cross linked cellulose such as croscarmellose sodium, for example, Ac-Di-Sol SD-711 (FMC Corporation), Primellose® and Vivasol®, and cross linked polyvinyl N-pyrrolidones such as crospovidone (Kollidon®) and Polyplasdone™.

In some embodiments, the disintegrant is selected from sodium starch glycolate, low-substituted hydroxy propyl cellulose and sorbitol or mixtures of two or more of these disintegrants.

In some embodiments, the composition comprises one disintegrant. In other embodiments, the composition comprises more than one disintegrant. In some embodiments the composition comprises a superdisintegrant or a mixture of superdisintegrant(s) and/or disintegrant(s).

In other embodiments, the disintegrant or superdisintegrant or mixture thereof is present in an amount of about 1% to about 25% w/w of the composition, especially about 4% to about 25% w/w of the composition, even more especially about 15% to about 25% w/w of the composition.

In some embodiments the disintegrant comprises a mixture of LHPC and sodium starch glycolate, preferably in equal ratios.

Suitable diluents may be one or more selected from the group consisting of a sugar, such as sucrose (e.g., Dipac®), glucose, dextrose, molasses, mannitol, sorbitol, xylitol (e.g., Xylitab®), lactose (e.g., lactose monohydrate, lactose anhydrous, etc.); alginic acid and salts thereof, such as sodium alginate; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), microcrystalline cellulose (e.g., Avicel®); silicified microcrystalline cellulose; microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; dextrin, dicalcium phosphate; a natural or synthetic gum such as agar, guar, locust bean, Karaya, pectin, tragacanth, acacia or ghatti gum, polyvinylpyrrolidone (e.g., Polyvidone® CL, Kollidon® CL, Polyplasdone® XL-10), larch arabogalactan, a clay such as Veegum® HV (magnesium aluminum silicate), polyethylene glycol, waxes, a starch, e.g., a natural starch such as corn starch or potato starch, a pregelatinized starch such as Colorcon (Starch 1500), National 1551 or Amijel®, or sodium starch glycolate such as Primogel® or Explotab®; a cross-linked starch such as sodium starch glycolate; a cross-linked polymer such as crospovidone; a cross-linked polyvinylpyrrolidone, a natural sponge; a surfactant; a resin such as a cation-exchange resin; citrus pulp; sodium lauryl sulfate; sodium lauryl sulfate in combination with starch; and combinations thereof. In a particular embodiment, the diluent is a sugar, such as sucrose (e.g., Dipac®), glucose, dextrose, molasses, lactose (e.g., lactose monohydrate, lactose anhydrous, etc.), a sugar alcohol such as mannitol, sorbitol, xylitol (e.g., Xylitab.®), especially where the sugar is mannitol. A suitable mannitol product is Pearlitol® 100SD (Roquette).

In some embodiments, the diluent is present in an amount of from about 1% to about 92% w/w of the composition, especially about 5% to about 85% or about 5% to about 75% or about 10% to 60% w/w, more especially about 20% to 50% w/w of the composition and even more especially 30% to 40% of the composition.

Lubricants are broadly described as “an additive to reduce friction”. Lubricants prevent ingredients from clumping together and from sticking to the tablet punches or capsule filling machine.

Lubrication is considered an essential in tablet and capsule manufacturing, firstly to reduce friction between the surfaces of manufacturing equipment and that of organic solids, and secondly the improve the powder flow by reducing the friction between the particles of the blend (inter-particle friction). In the later circumstances, glidants are also a form of lubricant and can be included in this list. The first type of friction is commonly called wall friction and the second type is internal friction or cohesion. Each of these require lubrication to overcome sticking and improve powder flowability.

Suitable lubricants may be one or more selected from the group consisting of stearic acid or salts thereof such as sodium stearate, magnesium stearate and zinc stearate, calcium hydroxide, talc, corn starch, sodium stearyl fumarate, alkali-metal and alkaline earth metal salts, such as aluminum, calcium, magnesium, zinc, waxes, Stearowet®, boric acid, sodium benzoate, sodium acetate, sodium chloride, leucine, a polyethylene glycol such as Carbowax, PEG 4000, PEG 5000, PEG 6000, propylene glycol, sodium oleate, glyceryl behenate, glyceryl palmitostearate, glyceryl benzoate, magnesium or sodium lauryl sulfate, and combinations thereof. In some embodiments, the lubricant is stearic acid or a salt thereof, especially magnesium stearate, zinc stearate or sodium stearate, more especially magnesium stearate.

In some embodiments, the lubricant is present in an amount of about 0.1% to about 5% w/w of the composition especially about 0.5% to about 2% w/w of the composition, especially about 1% w/w of the composition.

In some embodiments, the pharmaceutical compositions may further comprise other excipients such as binders, alkalizing agents and the like.

In particular embodiments of the pharmaceutical compositions of the invention, the ratio of peanut flour to adsorbent is in the range of 1:0.35 to 1 to 0.7, for example, 1:0.35, 1:0.4, 1:0.45, 1:0.5, 1:0.55, 1:0.6, 1:0.65 and 1:0.7 and all ratios in between.

In particular embodiments of the pharmaceutical compositions of the invention, the ratio of peanut protein to adsorbent is in the range of 0.5:0.35 to 0.5 to 0.7, for example, 0.5:0.35, 0.5:0.4, 0.5:0.45, 1:1, 0.5:0.55, 0.5:0.6, 0.5:0.65 and 0.5:0.7 and all ratios in between.

The ratio of adsorbent to peanut flour is maintained in the composition and the amount of adsorbent is calculated from the amount of peanut flour used. Any difference in amounts of each component on a batch to batch basis is accounted for by increasing or decreasing the amount of diluent present in the composition.

The pharmaceutical compositions herein may be formulated for use in oral administration. In some embodiments, the pharmaceutical composition is formulated to provide a granule, a mini-tablet, a tablet, an oro-dispersible tablet, a powder or a sprinkle formulation. In particular embodiments, the pharmaceutical composition is formulated into a granule, tablet or mini-tablet formulation as such formulations prevent peanut flour becoming airborne (because it is compressed and only disintegrates when contacts food), which reduces potential for peanut allergens to be inhaled. Inhalation of peanut flour represents a risk for an anaphylaxis reaction in allergic individuals. In particular granule, tablet or mini-tablet formulations are highly desirable for treatment of pediatric or elderly populations who may have difficulty consuming tablets or capsules and find it difficult to open capsules containing powdered peanut flour or peanut protein.

The term “mini-tablets” commonly refers to compressed tablets with size smaller than typical tablets. Mini-tablets typically have diameters between one to four millimeters (mm). Since oral dosage forms smaller than 2.5 mm can be considered as oral granules, many mini-tablet products are focused at this size range, to take advantage of the potential flexibility in dosage form administration (e.g. mixed with soft foods) Granules can be dissolved in soft foods or beverages and are associated with better dosage compliance in pediatric populations than other solid form dosage forms.

Mini-tablets are generally manufactured by direct compression using a rotary tablet press. For example, the mini-tablets may be manufactured in a process in which the peanut flour is first blended with the adsorbent and in a second step all of the remaining excipients (with the exception of the lubricant) are blended. Finally, the lubricant is added and following blending prior to direct compression using a rotary tablet press.

In another aspect of the present invention there is provided a method of making the composition of the invention comprising the steps of:
a. blending peanut flour with at least one adsorbent; wherein the peanut flour comprises 40% to 60% peanut protein;
b. mixing at least one diluent and at least one disintegrant with the blend from step a;
c. adding at least one lubricant to the mixture of step b. and mixing.

Any further excipients may be added in step b.

In some embodiments, the method further comprising formulating the mixture from step c. into granules, tablets or mini-tablets.

A pharmaceutical composition according to the present invention has acceptable flow and binding properties such that it may be compressed into the granule, tablet or mini-tablet, acceptable handling properties during manufacture and packaging and acceptable disintegration times when mixing with water or soft foods or the ability to be crushed to aid in disintegration in water or soft foods.

Key attributes used to assess each composition are as follows:
Flowability of the bulk formula—the bulk formula needs to be able to flow easily from a hopper into a tablet press where the bulk formula is compressed into granules, tablets or mini-tablets;
Compression of the granules—need to be able to compress the bulk formula to form granules, tablets or mini-tablets in the tablet press; and
Hardness of the granules—the granules, tablets or mini-tablets need to be hard enough to survive moving from the tablet press to where they are packaged, i.e. not fall apart or break.

The disintegration time of the granules, tablets or mini-tablets in water (USP<701>, United States Pharmacopeia General Chapter 701 Disintegration) and a soft food within 2 minutes may also be an important attribute of the composition.

In some embodiments, the granule, tablet or mini-tablet is formulated to have a tablet hardness of at least 4N and particularly between 4N to 15N, especially 4 to 8N, more especially 4 to 5 N, when measured using a Multitest 50 Tablet Hardness tester. It is important to balance the hardness of the tablet with its ease of disintegration and/or crushing.

The size of the granule, tablet or mini-tablet may vary, however, for amounts of peanut protein in the range of 0.1 mg to 3 mg, the granule, tablet or mini-tablet may have a size in the range of from 1.5 mm to 3.5 mm, especially about 2.0 mm to 2.5 mm.

In some embodiments, the granule, tablet or mini-tablet is formulated such that it is dispersed in water, a solution or a food product.

In some embodiments, the solution is an aqueous solution, for example, the granule, tablet or mini-tablet may be dispersed in water or a flavored drink such as a cordial or Kool-Aid, or milk or flavored milk. In such embodiments, it is preferred if the granule, tablet or mini-tablet is able to disperse into the solution in less than 5 minutes, for example, in less than 4.5 minutes, less than 4 minutes, less than 3.5 minutes or less than 3 minutes and especially less than 2 minutes. In some embodiments, the composition disperses into the solution in between 10 seconds and 3 minutes, especially 10 seconds and 2.5 minutes and more especially between 10 seconds and 2 minutes.

Any of the compositions provided herein may be dispersed in a food product. A food product as provided herein may be any food with which the composition may be mixed for consumption. In some embodiments, composition may be added to a soft food product in which it is able to at least partially dissolve or disperse, for example, a puree or sauce, such as a fruit puree, vegetable puree, fruit sauce or vegetable sauce or a soft pudding, such as a custard. In particular embodiments, granules, tablets or mini-tablets of the pharmaceutical composition may completely dissolve or disperse in the food product. In other embodiments, the granules, tablets or mini-tablets may be sprinkled on a food and consumed without dissolution or dispersion or with only partial dissolution or dispersion.

In some embodiments, the granule, tablet or mini-tablet formulation comprises an amount of peanut protein in the range of from 0.1 mg to 10 mg per granule, tablet or mini-tablet, especially 0.1 mg to 5 mg, more especially 0.4 mg to 4 mg. In some embodiments, the granule, tablet or mini-tablet formulation comprises about 0.1 mg, 0.4 mg or 2.0 mg of peanut protein per granule, tablet or mini-tablet.

The compositions of the invention, particularly the granule, tablet or mini-tablet formulations, may be presented in any convenient form for example, filled into capsules, or filled into a sachet or stick pack. Advantageously, each of these formulations allow for dose flexibility and the delivery of a quantified dosage amount of peanut protein. For example, a sachet or stick packet may contain 10 granules or mini-tablets that each contain 2 mg of peanut protein, providing a dosage of 20 mg of peanut protein. Dosages may be varied by varying the number of granules or mini-tablets included in the package and/or the amount of peanut protein in each granule.

The present invention provides a composition or formulation for the treatment of peanut allergy, optionally by oral immunotherapy.

In one aspect of the invention there is provided a method of treating peanut allergy comprising administering a pharmaceutical composition of the invention, optionally by oral immunotherapy.

In a further aspect of the invention, there is provided a composition of the invention for use in treating peanut allergy, optionally by oral immunotherapy.

In yet a further aspect of the invention there is provided a use of peanut flour, at least one disintegrant, at least one adsorbent, at least one diluent and at least one lubricant, wherein the peanut flour comprises 40 to 60% peanut protein, in the manufacture of a medicament for treating peanut allergy, optionally by oral immunotherapy.

“Treating” or “treatment” as used herein means any improvement in the peanut allergy. It includes alleviating at least one symptom, or reducing the risk, occurrence or severity of allergic response. In a particular embodiment, it includes desensitizing the subject, achieving sustained unresponsiveness or achieving tolerance to a particular allergen. “Desensitization” refers to a transient increase in the amount of allergen a subject is able to tolerate without reaction (e.g. at the level of exposure during the OIT, e.g. the amount used during the maintenance phase, or at an increased level) that is lost when immunotherapy or regular allergen exposure is discontinued. “Sustained unresponsiveness” as used herein describes the sustained ability of a subject to tolerate an allergen (e.g. at the level of exposure during the OIT, e.g. the amount used during the maintenance phase, or at an increased level) after stopping immunotherapy for a period of at least 2-4 weeks without requiring regular and frequent exposure to the antigen (e.g. as might be required if a subject is merely desensitized to an allergen). “Tolerance” as used herein, by contrast, refers to the long-lasting (e.g. years) or permanent ability of a subject to ingest any amount of an allergen via the oral route without reaction, which does not require continued allergen exposure to maintain such a state. In certain embodiments, the present invention therefore provides the treatment of an allergy to a peanut allergen which provides sustained unresponsiveness or tolerance, whereas in other embodiments, it may provide desensitization of a subject to a peanut allergen to which they are allergic.

“Allergy” as used herein refers to acquired hypersensitivity to an allergen. Subjects having an allergy to an allergen may be referred to as allergic subjects, and these terms are used interchangeably herein. The invention is concerned solely with peanut allergy. “Peanut allergy” is an adverse response to a peanut allergen triggered by an immunological reaction to the allergen. A peanut allergy may also be considered an allergy to a peanut allergen. A peanut allergy should be distinguished from non-immune-mediated adverse responses to peanut, such as intolerance and toxin-mediated reactions (e.g. to aflatoxin).

An individual with an allergy to peanut allergen(s) may display peanut allergen-specific serum IgE, i.e. IgE which specifically binds to the peanut allergen or may have non-IgE mediated allergy wherein the allergy is not mediated primarily by the allergen-specific IgE. Patients may be diagnosed with the peanut allergy according to standard clinical criteria. Standard clinical criteria may include for example, a history of a type-1 hypersensitivity reaction which is temporally related to peanut allergen ingestion (e.g. hives, swelling, wheezing, abdominal pain, vomiting, breathlessness, hypotension, collapse), and the presence of peanut allergen-specific IgE by positive skin prick test (wheal diameter>/=3 mm or >/=2 mm in infants) or ImmunoCap serum IgE>0.35 kUA/L in the case of IgE-mediated allergy or based on history of delayed symptoms to allergen ingestion (e.g. abdominal pain, vomiting, diarrhea, malabsorption, weight loss, failure to thrive, eczema, eosinophilic esophagitis, eosinophilic gastroenteritis, food protein induced proctocolitis, food protein induced enterocolitis syndrome (FPIES)) with or without abnormal findings on endoscopy and biopsy in the case of mixed IgE/non-IgE mediated food allergy or non-IgE mediated food allergy.

As used herein an “oral immunotherapy regime” or OIT refers to the treatment regime to be used for the treatment of peanut allergy. This treatment regime comprises the oral administration of peanut allergen in increasing doses over weeks, months or years with the aim of stimulating the immune system to develop desensitization, sustained unresponsiveness or tolerance with the aim of alleviating an allergic response.

OIT protocols typically include three phases of treatment—rush (also referred to as initiation), build-up and maintenance. Together the rush phase and build-up phase form the dose escalation phase. The rush phase generally involves administering a series of escalating allergen doses (e.g. 6-8 doses) over one day, starting at a very low dose that the subject can tolerate, often as low as 0.1 mg. The build-up phase generally involves administering a daily dose of the food allergen, typically commencing at the highest tolerated dose of the rush phase (or the cumulative tolerated dose in the rush phase if all rush phase doses are tolerated), with increases in the dose every 1-2 weeks. The maintenance phase continues the treatment at the highest dose reached during build-up phase and provides the same dose of the food allergen daily, often for months to years or indefinitely. The highest dose may be 1000 mg or 2000 mg or 4000 mg or more.

The pharmaceutical compositions of the present invention allow for the different doses of peanut allergen to be very accurate, which is particularly important, especially in the dose escalation phase when the subject is particular susceptible to small variations in dose.

Increasing the dose in increments allows the dose to be increased gradually over time, thereby minimizing the risk that subjects suffer an adverse event to the allergen. Without wishing to be bound by theory, it is believed that as a subject is exposed to increasing doses of the peanut allergen to which they are allergic, they are capable of tolerating gradually increasing doses of the allergen. Thus, an increase from a tolerated dose of the allergen is less likely to cause an adverse event during a dose escalation phase.

By way of representative example, a dose escalation phase may comprise a series of dose increases whereby the dose of the allergen is increased every 30 minutes for up to 12 hours, followed by increases in the dose of the allergen every two weeks (e.g. 10 to 18 days).

It has previously been found by the present inventors that addition of a probiotic to peanut OIT may provide benefits in an OIT dosing regime (referring to WO 2009/094717). Without wishing to be bound by theory it is believed that the induction of plasmacytoid dendritic cells (pDCs) by such probiotics inhibits the allergic response to the peanut allergen (that is administered in the OIT treatment), improving the tolerability of the treatment. One way this could be achieved is by pDCs supporting the differentiation of allergen-specific Treg cells, which in turn suppress the development of allergic reactions caused by exposure to peanut allergen during OIT. Treg may also inhibit intestinal epithelial inflammation and improve gut barrier function, thereby limiting the gastrointestinal symptoms caused by peanut allergen exposure during OIT.

In some embodiments the OIT may further comprise the additional administration of a probiotic. A “probiotic” as used herein refers to a microorganism that is used in the cure, mitigation, treatment, or prevention of disease. The probiotic may stimulate the production of tolerogenic DCs, or in other words, an increased number of tolerogenic DCs may be induced by or in response to the probiotic. The induction occurs in vivo but may be tested in vivo or in vitro. Preferably, the tolerogenic DC is a pDC. A wide variety of probiotics including Lactobacillus rhamnosus, Bifidobacterium lactis, Saccharomyces boulardi, E. coli Nissle 1917, Streptococcus thermophilus and Bifidobacterium breve induce pDCs, and these probiotics represent particularly preferred probiotics for use in the treatments of the present invention. The ability to induce pDCs is similar in allergic subjects and subjects with allergic disease.

In further embodiments, the probiotic may be any probiotic microorganism that is a species of Lactobacillus, Bifidobacterium, Escherichia, Saccharomyces, Streptococcus or Bacillus. In a particularly preferred embodiment, the species is from Lactobacillus, Bifidobacterium, Saccharomyces or Streptococcus.

In some embodiments, the probiotic is a bacterial species selected from the group consisting of Clostridium bolteae, Anaerotruncus colihominis, Sellimonas intestinales, Clostridium symbiosum, Blautia producta, Dorea longicatena, Erysipelotrichaceae bacterium, Subdolinogranulum spp. Ruminococcus torques, Clostridium innocuum, Flavinofractor plautii, and combination thereof.

In embodiments, the probiotic may be Eubacterium rectale, Clostridium ramosum, Butyrovibrio crossatus, Roseburia intestinalis, Clostridium hylemonae, Hungatella hathawayi, Clostridium symbiosum, Faecalibacterium prausnitzii, Subdoligranulum variabile, Bacteroides spp., Bacteroides thetaiotaomicron, Bacteroides fragilis, Bacteroides ovatus, Parabacetroides goldsteinii, Parabacteroides merdae, Parabacteroides distasonis, Prevotella tannerae, Clostridium sardiniensis, Clostridium hiranonsis, Facealibacterium prausnitzii, Butyrovibrio spp., Eubacterium rectale, Roseburia intestinalis, Clostridium scindens, Clostridium spp (e.g., Clostridium ramosum, Clostridium scindens, Clostridium hiranonsis, Clostridium bifermentans, Clostridium leptum, Clostridium sardiniensis, Clostridium hathewayi, Clostridium nexile, Clostridium hylemonae, Clostridium glycyrrhizinilyticum, Clostridium lavalense, Clostridium fimetarium, Clostridium symbiosum, Clostridium sporosphaeroides etc.), Prevotella copri, Prevotella paludivivens, and combinations thereof.

In one embodiment the probiotic microorganism is a species of Lactobacillus, selected from the list of Lactobacillus acidophilus, Lactobacillus casei, Lactobacillus johnsonii, Lactobacillus lactis, Lactobacillus plantarum, Lactobacillus reuteri, Lactobacillus rhamnosus, Lactobacillus salivarius and Lactobacillus helveticus.

In a particularly preferred embodiment, the probiotic is Lactobacillus rhamnosus, and in particular may be Lactobacillus rhamnosus GG. In other embodiments, the LBP or probiotic may be Bifidobacterium lactis, Saccharomyces boulardi, E. coli Nissle 1917, Streptococcus thermophilus or Bifidobacterium breve.

According to preferred embodiments, the pharmaceutical composition may comprise the probiotic or may be administered separately, simultaneously or sequentially.

In one embodiment the pharmaceutical composition and the probiotic are administered simultaneously.

According to certain embodiments of the present invention, when the probiotic and the pharmaceutical composition are administered in separate formulations, the pharmaceutical composition may be administered one minute, two minutes, three minutes, four minutes, five minutes, ten minutes, 30 minutes, 1 hour, 2 hours, 3 hours, 4 hours, 5 hours, 6 hours, 7 hours, 8 hours, 9 hours, 10 hours, 11 hours or more up to 12 hours before the probiotic. According to alternative embodiments, the probiotic may be administered one minute, two minutes, three minutes, four minutes, five minutes, ten minutes, 30 minutes, 1 hour, 2 hours, 3 hours, 4 hours, 5 hours, 6 hours, 7 hours, 8 hours, 9 hours, 10 hours, 11 hours or more up to 12 hours before the pharmaceutical composition.

According to yet further embodiments of the present invention, the separate and/or sequential administration of the probiotic and the pharmaceutical composition may comprise administering each of the two components separated by greater than 12 hours. Thus, according to certain embodiments, the pharmaceutical composition and the probiotic may be administered separately up to 7 days apart, more preferably up to 6, 5, 4, 3, 2 or 1 days apart. In certain embodiments, the probiotic may be administered up to 7, 6, 5, 4, 3, 2 or 1 day before the pharmaceutical composition. According to other embodiments, the pharmaceutical composition may be administered up to 7, 6, 5, 4, 3, 2, or 1 day before the probiotic.

The probiotic is to be administered at a dose sufficient to allow safe transit of sufficient probiotic through a subject's stomach that their beneficial effects may be felt by the subject. Probiotics are emerging dietary supplement and pharmaceutical products, and doses of probiotics which are administered to allow probiotics to reach the gut in suitable numbers to provide health benefits to a subject are known and may vary accordingly between probiotics. Suitable doses may be any one of 1×10⁶, 5×10⁶, 1×10⁷, 5×10⁷ or 1×10⁸CFU to any one of 5×10⁸, 1×10⁹, 5×10⁹, 1×10¹⁰, 2×10¹⁰, 5×10¹⁰, 1×10¹¹ or 1×10¹² CFU, for example 1×10⁶ to 1×10¹², 5×10⁶ to 1×10¹¹, 1×10⁷ to 5×10¹⁰, 1×10⁸ to 2×10¹⁰, 1×10⁶-1×10⁸, 1×10⁷-1×10⁹, 1×10⁸-1×10¹⁰, 1×10⁹-1×10¹¹ or 1×10¹⁰-1×10¹². However, doses of probiotics in the range of 1×10⁸ CFU to 2×10¹⁰ CFU are typically advised, and such doses of probiotics may preferably be administered in the treatments of the present invention. Doses within this range may, therefore, be administered at least weekly, or at least every three days, two days, or preferably daily, to subjects in accordance with the treatment of the present invention. In one embodiment the same dose of probiotic is used for every administration. However, in other embodiments the doses may optionally vary, for example the probiotic dose may increase during the course of the treatment, e.g. in line with increases made to the dose of peanut protein in the pharmaceutical composition.

The probiotic may also be administered by way of a food product, or more preferably may be administered in a solution for consumption by drinking. The probiotic may therefore preferably be administered in water, cow's milk or soy milk, or other milk or drink. The probiotic may be provided as set doses e.g. as a fixed dose in a sachet or stickpack that may be mixed with a drink or food, within capsules or in a tub with a standardized measuring scoop.

Additional agents may be used during the treatment to support the treatment. For example, the pharmaceutical composition may be administered with a prebiotic which is known to support the growth of probiotic organisms. Alternatively, the prebiotic may be administered without any probiotic to support the growth of probiotic organisms already present in the gut of the subject being treated.

In further embodiments other agents such as anti-allergy drugs, such as antihistamines, steroids, bronchodilators, leukotriene stabilizers and mast cell stabilizers may be used during the OIT treatment. Suitable anti-allergy drugs are well known in the art. Such agents may be useful in reducing allergic inflammation and increasing tolerance of the allergen.

The subject referred to herein refers to an animal, particularly a mammal and more particularly a human with an allergy to an allergen, who can benefit from the treatments of the present invention. In a particularly preferred embodiment, the subject may be an adult. However preferably, the subject is not an adult, and in certain embodiments the subject may be under 18 years old, i.e. the subject may be a child. Yet more preferably, the subject may be a child under 12 years old or may be a child under 6 years old. In a yet further embodiment, the subject may be from 6 years old, e.g. to 12 years old or 18 years old, and in another embodiment may be from 5 years old to 10 years old. In a particularly preferred embodiment, the child may be from 1 year to 18 years old.

The methods described herein may be used for any patient with peanut allergy and are independent of the patient's sensitivity or challenge threshold to allergen, the weight or height of the patient and other factors.

In another aspect of the present invention there is provided a kit comprising at least two doses of the composition of the invention described above, each dose packaged separately.

In some embodiments, the kit contains multiple doses of the composition of the invention, for example, sufficient doses to complete a dose escalation phase or to complete at least part of a maintenance phase.

In some embodiments, the multiple doses of the composition of the invention contain the same amount of peanut protein. In other embodiments, the multiple doses of composition contain different doses of peanut protein, for example, increasing doses of peanut protein, suitable for use in a dose escalation phase.

In some embodiments each dose unit in the kit is in the form of a granule or mini-tablet formulation optionally packaged in a sachet or stick packet.

In some embodiments, the kit further comprises one or more doses of a probiotic and/or prebiotic.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

In addition, as used herein, the term “about” refers to a quantity, level, value, dimension, size, or amount that varies by as much as 15%, 10% or 5% to a reference quantity, level, value, dimension, size, or amount.

It is to be understood that, if any prior art publication is referred to herein, such reference does not constitute an admission that the publication forms a part of the common general knowledge in the art, in Australia or any other country.

The invention will now be described by way of non-limiting Examples.

### EXAMPLES

### Example 1: Composition 1 (Formulation C)—2 mg Peanut Protein Per Granule

Granules were prepared using the following ingredients:

| | Mg/ | % w/w of |
|---|---|---|
| Ingredient | granule | granule |
| Peanut Flour | 4.000* | 54.05 |
| Mannitol 100 SD | 0.868 | 11.73 |
| Silicon dioxide (Syloid) | 2.082 | 28.14 |
| Sodium starch glycolate | 0.370 | 5.0 |
| Magnesium stearate NF Hyqual | 0.080 | 1.08 |
| Total | 7.40 | 100.0 |

*Potency adjusted to 2 mg protein based on the protein content of each batch of peanut flour

The following steps were performed for the manufacture of a batch of granules:
1. the quantity of peanut flour required based on the protein content of the batch of peanut flour (potency adjustment) was calculated. The quantity of mannitol required is adjusted based on the peanut flour quantity.
2. the raw materials for manufacture were dispensed,
3. the peanut flour and silicon dioxide were mixed together until blended.
4. mannitol, L-HPC, Sodium Starch Glycolate and sorbitol was added to the peanut flour and silicon dioxide blend and mixed.
5. magnesium stearate was added to the blended ingredients and mixed with care needed not to over mix the lubricant to avoid loss of integrity in the formulation.
6. the granules were compressed using tooling to achieve target size and to the target compression force.
7. The granules were then collected and sampled to test for hardness, weight, thickness and disintegration.

The equipment used in the testing was as follows:

Hardness—Multitest 50 Tablet Hardness tester. This equipment can also measure the weight and thickness of the tablet/granule

Disintegration—Erweka ZT 72 Automatic Disintegration Tester. Disintegration in water is tested at 37° C. and for soft food, at room temperature.

### Example 2: Composition 2 (Formulation D)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 42.55 |
| Mannitol 100 SD | 1.208 | 12.85 |
| Silicon dioxide (Syloid) | 3.132 | 33.32 |
| Low-substituted hydroxy propyl cellulose (L-HPC) | 0.480 | 5.11 |
| Sodium starch glycolate | 0.480 | 5.11 |
| Magnesium stearate NF Hyqual | 0.100 | 1.064 |
| Total | 9.40 | 100.0 |

A compression hardness of 9.7N was achieved

### Example 3: Composition 3 (Formulation G)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 42.55 |
| Mannitol 100 SD | 1.195 | 12.71 |
| Silicon dioxide (Syloid) | 2.525 | 26.86 |
| Low-substituted hydroxy propyl cellulose (L-HPC) | 0.480 | 5.11 |
| Sodium starch glycolate | 0.480 | 5.11 |
| Sorbitol (Neosorb P60W) | 0.620 | 6.60 |
| Magnesium stearate NF Hyqual | 0.100 | 1.06 |
| Total | 9.40 | 100.0 |

A compression hardness of 9N was achieved.

### Example 4: Composition 4 (Formulation I)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 44.94 |
| Mannitol 100 SD | 1.280 | 14.38 |
| Silicon dioxide (Syloid) | 2.120 | 23.82 |
| Low-substituted hydroxy propyl cellulose (L-HPC) | 0.700 | 7.87 |
| Sodium starch glycolate | 0.700 | 7.87 |
| Magnesium stearate NF Hyqual | 0.100 | 1.12 |
| Total | 8.90 | 100.0 |

A compression hardness of 8-10 N was achieved.

### Example 5: Composition 5 (Formulation J)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 47.06 |
| Mannitol 100 SD | 0.918 | 10.80 |
| Silicon dioxide (Syloid) | 3.132 | 36.85 |
| Sodium starch glycolate | 0.370 | 4.35 |
| Magnesium stearate NF Hyqual | 0.080 | 0.94 |
| Total | 8.50 | 100.0 |

A compression hardness of 4-5N was achieved.

### Example 6: Composition 6 (Formulation K)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 51.95 |
| Mannitol 100 SD | 0.870 | 11.30 |
| Silicon dioxide (Syloid) | 2.120 | 27.53 |
| Sodium starch glycolate | 0.630 | 8.18 |
| Magnesium stearate NF Hyqual | 0.080 | 1.04 |
| Total | 7.70 | 100.0 |

A compression hardness of 4-5N was achieved.

### Example 7: Composition 7 (Formulation L)—2 mg Peanut Protein Per Granule

Granules were prepared using the following ingredients:

| | Mg/ | % w/w of |
|---|---|---|
| Ingredient | granule | granule |
| Peanut Flour | 4.000 | 42.11 |
| Mannitol 100 SD | 1.200 | 12.63 |
| Silicon dioxide (Syloid) | 2.120 | 22.32 |
| Low-substituted hydroxy propyl cellulose (L-HPC) | 0.730 | 7.68 |
| Sodium starch glycolate | 0.730 | 7.68 |
| Sorbitol (Neosorb P60W) | 0.620 | 6.53 |
| Magnesium stearate NF Hyqual | 0.100 | 1.05 |
| Total | 9.50 | 100.0 |

A compression hardness of 4-5N was achieved.

### Example 8: Composition 8 (Formulation M)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 45.71 |
| Mannitol 100 SD | 1.210 | 13.83 |
| Silicon dioxide (Syloid) | 2.120 | 24.23 |
| Sodium starch glycolate | 0.700 | 8.0 |
| Sorbitol (Neosorb P60W) | 0.620 | 7.09 |
| Magnesium stearate NF Hyqual | 0.100 | 1.14 |
| Total | 8.75 | 100.0 |

A compression hardness of 4-5N was achieved.

### Example 9: Composition 9 (Formulation N)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 44.20 |
| Mannitol 100 SD | 1.195 | 13.20 |
| Silicon dioxide (Syloid) | 1.675 | 18.51 |
| Low-substituted hydroxy propyl cellulose (L-HPC) | 0.730 | 8.07 |
| Sodium starch glycolate | 0.730 | 8.07 |
| Sorbitol (Neosorb P60W) | 0.620 | 6.85 |
| Magnesium stearate NF Hyqual | 0.100 | 1.10 |
| Total | 9.05 | 100.0 |

Compression hardness of 4N and 8N were achieved.

### Example 10: Composition 10 (Formulation O)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 46.30 |
| Mannitol 100 SD | 1.195 | 13.83 |
| Silicon dioxide (Syloid) | 1.265 | 14.64 |
| Low-substituted hydroxy propyl cellulose (L-HPC) | 0.730 | 8.45 |
| Sodium starch glycolate | 0.730 | 8.45 |
| Sorbitol (Neosorb P60W) | 0.620 | 7.18 |
| Magnesium stearate NF Hyqual | 0.100 | 1.16 |
| Total | 8.64 | 100.0 |

A compression hardness of 3-3.5N was achieved.

### Example 11: Composition 11 (Formulation P)—2 mg Peanut Protein Per Granule

Granules were prepared using the method of Example 1 with the following ingredients:

| | Mg/ | % w/w of |
|---|---|---|
| Ingredient | granule | granule |
| Peanut Flour | 4.000 | 45.20 |
| Mannitol 100 SD | 1.195 | 13.50 |
| Silicon dioxide (Syloid) | 1.475 | 16.67 |
| Low-substituted hydroxy propyl cellulose (L-HPC) | 0.730 | 8.25 |
| Sodium starch glycolate | 0.730 | 8.25 |
| Sorbitol (Neosorb P60W) | 0.620 | 7.01 |
| Magnesium stearate NF Hyqual | 0.100 | 1.13 |
| Total | 8.85 | 100.0 |

A compression hardness of 4-4,5N was achieved.

### Example 12: Composition 12 (Formulation A 0.1 mg)—0.1 mg Peanut Protein Per Granule

Granules were prepared using the method of Example 1 with the following ingredients:

| | Mg/ | % w/w of |
|---|---|---|
| Ingredient | granule | granule |
| Peanut Flour | 0.200 | 3.08 |
| Mannitol 100 SD | 4.540 | 69.85 |
| Colloidal silicon dioxide and microcrystalline | 1.500 | 23.08 |
| cellulose (Prosolv SMCC 50) | | |
| Croscarmellose sodium (Ac-Di-Sol SD-711) | 0.200 | 3.08 |
| Magnesium stearate NF Hyqual | 0.060 | 0.92 |
| Total | 6.500 | 100.0 |

A compression hardness of 12N was achieved.

### Example 13: Composition 13 (Formulation B 0.1 mg)—0.1 mg Peanut Protein Per Granule

Granules were prepared using the method of Example 1 with the following ingredients:

| | Mg/ | % w/w of |
|---|---|---|
| Ingredient | granule | granule |
| Peanut Flour | 0.200 | 3.08 |
| Mannitol 100 SD | 5.515 | 84.85 |
| Colloidal silicon dioxide and microcrystalline | 0.525 | 8.08 |
| cellulose (Prosolv SMCC 50) | | |
| Sodium starch glycolate | 0.200 | 3.08 |
| Magnesium stearate NF Hyqual | 0.060 | 0.92 |
| Total | 6.500 | 100.0 |

A compression hardness of 14.8N was achieved.

### Example 14: Composition 14 (Formulation A 0.4 mg)—0.4 mg Peanut Protein Per Granule

Granules were prepared using the method of Example 1 with the following ingredients:

| | Mg/ | % w/w of |
|---|---|---|
| Ingredient | granule | granule |
| Peanut Flour | 0.800 | 16.16 |
| Mannitol 100 SD | 2.62 | 52.93 |
| Silicon dioxide (Syloid) | 0.335 | 6.77 |
| Low-substituted hydroxy propyl cellulose (L-HPC) | 0.400 | 8.08 |
| Sodium starch glycolate | 0.400 | 8.08 |
| Sorbitol (Neosorb P60W) | 0.34 | 6.87 |
| Magnesium stearate NF Hyqual | 0.055 | 1.11 |
| Total | 4.950 | 100.0 |

A compression hardness of 3.2N was achieved

### Example 15: Composition 15 (Formulation B 0.4 mg)—0.4 mg Peanut Protein Per Granule

Granules were prepared using the method of Example 1 with the following ingredients:

| | Mg/ | % w/w of |
|---|---|---|
| Ingredient | granule | granule |
| Peanut Flour | 0.800 | 16.16 |
| Mannitol 100 SD | 2.58 | 49.61 |
| Silicon dioxide (Syloid) | 0.585 | 11.25 |
| Low-substituted hydroxy propyl cellulose (L-HPC) | 0.400 | 7.69 |
| Sodium starch glycolate | 0.400 | 7.69 |
| Sorbitol (Neosorb P60W) | 0.34 | 6.54 |
| Magnesium stearate NF Hyqual | 0.055 | 1.06 |
| Total | 5.200 | 100.0 |

A compression hardness of 4.0N was achieved

### Example 16: Disintegration Testing

The compositions of the invention were disintegration tested according to the method described in United States Pharmacopeia General Chapter 701—Disintegration.

The results are given in the table below. As can be seen from the formulations tested there is a delicate balance between flowability, hardness and disintegration time which is only obtained by certain formulations and can vary dependent on the amount of peanut protein (granule strength).

Formulations per strength and attributes in order of development

| Granule Strength | | | | Granule | | Disintegration | Disintegration |
|---|---|---|---|---|---|---|---|
| (peanut protein) | | Formulation No. | Flowability | Compression | Hardness | water | soft food |
| 0.1 | mg | A | ✓ | ✓ | ✓ | ✓ | NT |
| | | B | ✓ | ✓ | ✓ | ✓ | NT |
| 0.83 | mg | A | X | X | NT | NT | NT |
| 2.0 | mg | A | X | X | NT | NT | NT |
| | | B | X | X | NT | NT | NT |
| | | C | ✓ | ✓ | ✓ | X | X |
| | | D | ✓ | ✓ | ✓ | X | X |
| | | E | X | X | X | NT | NT |
| | | F | ✓ | ✓ | ✓ | ✓ | X |
| | | G | ✓ | ✓ | ✓ | ✓ | X |
| | | H | ✓ | ✓ | ✓ | X | X |
| | | I | ✓ | ✓ | ✓ | ✓ | X |
| | | J | ✓ | ✓ | ✓ | ✓ | X |
| | | K | ✓ | ✓ | ✓ | ✓ | X |
| | | L | ✓ | ✓ | ✓ | ✓ | ✓ |
| | | M | ✓ | ✓ | ✓ | ✓ | X |
| | | N | ✓ | ✓ | ✓ | ✓ | ✓ |
| | | O | ✓ | ✓ | ✓ | ✓ | X |
| | | P | ✓ | ✓ | ✓ | ✓ | X |
| 0.4 | mg | A | ✓ | ✓ | ✓ | ✓ | NT |
| | | B | ✓ | ✓ | ✓ | ✓ | NT |

NT = Not tested
✓ acceptable
X not acceptable

1. A pharmaceutical composition comprising peanut flour, at least one disintegrant, at least one adsorbent, at least one diluent and at least one lubricant, wherein the peanut flour comprises about 40% to about 60% peanut protein.

2. The pharmaceutical composition according to claim 1, wherein the amount of peanut protein is between about 1% and about 60% w/w of the composition.

3. The pharmaceutical composition according to claim 1, wherein the amount of peanut protein is between about 1% and about 10% w/w or about 20% to about 40% w/w of the composition.

4. The pharmaceutical composition according to claim 1, wherein the ratio of peanut flour to adsorbent is in the range of 1:0.35 to 1:0.7 or the ratio of peanut protein to absorbent is in the range of 0.5:0.35 to 0.5:0.7.

5. The pharmaceutical composition according to claim 1, wherein the disintegrant is selected from sodium starch glycolate, croscarmellose sodium, crospovidone, low-substituted hydroxy propyl cellulose, sorbitol or a mixture of two or more of these disintegrants.

6. The pharmaceutical composition according to claim 1, wherein the disintegrant is present in an amount of about 1% to about 25% w/w of the composition.

7. The pharmaceutical composition according to claim 1, wherein the adsorbent is selected from synthetic amorphous silicon dioxide or silicon dioxide and microcrystalline cellulose.

8. The pharmaceutical composition according to claim 1, wherein the adsorbent is present in an amount of about 1% to about 40% w/w of the composition.

9. The pharmaceutical composition according to claim 1, wherein the diluent is a sugar selected from mannitol, sorbitol, xylitol, glucose, sucrose, dextrose, and lactose.

10. The pharmaceutical composition according to claim 1, wherein the diluent is present in an amount in the range of about 1% to about 92% w/w of the composition.

11. The pharmaceutical composition according to claim 1, wherein the lubricant is stearic acid or a salt thereof.

12. The pharmaceutical composition according to claim 1, wherein the amount of lubricant is in the range of about 0.1% to about 5% w/w of the composition.

13. The pharmaceutical composition according to claim 1 in the form of a granule, tablet or mini-tablet formulation.

14. The pharmaceutical composition according to claim 1, in the form of a, granule, tablet or mini-tablet formulation which has a hardness of at least 4N when measured by a Multitest 50 Tablet Hardness tester.

15. The pharmaceutical composition according to claim 13, wherein the granule, tablet or mini-tablet formulation has a disintegration time of 10 seconds to 3 minutes in water at 37° C. or in soft food at room temperature when measured by a Erweka ZT 72 Automatic Disintegration Tester.

16. A kit comprising at least two doses of the pharmaceutical composition of claim 1, each dose packaged separately.

17. The kit according to claim 16, further comprising a probiotic and/or a prebiotic.

18. A method of treating peanut allergy comprising administering the pharmaceutical composition of claim 1, optionally in an oral immunotherapy.

19. A method of making the pharmaceutical composition of claim 1, comprising the steps of:
a. blending peanut flour with at least one adsorbent; wherein the peanut flour comprises 40% to 60% peanut protein;
b. mixing at least one diluent and at least one disintegrant with the blend from step a;
c. adding at least one lubricant to the mixture of step b. and mixing.

20. The method according to claim 19 further comprising formulating the mixture from step c. into granules, tablets or mini-tablets.
